(19) United States
(12) Patent Application Publication
(10) Pub. No.: US 2012/0053182 A1
(43) Pub. Date: Mar. 1, 2012

(54) METHODS OF ALLEVIATING OR TREATING SIGNS AND/OR SYMPTOMS ASSOCIATED WITH MODERATE TO SEVERE PARKINSON'S DISEASE

(75) Inventors: Marc Cantillon, Livingston, NJ (US); Susan B. Huyck, Berkeley Heights, NJ (US); Kenneth P. Wolski, Carversville, PA (US)

(73) Assignee: SCHERING CORPORATION, Kenilworth, NJ (US)

(21) Appl. No.: 13/145,434

(22) PCT Filed: Jan. 13, 2010
(86) PCT No.: PCT/US2010/000063

§ 371 Date: Nov. 10, 2011

Related U.S. Application Data
(60) Provisional application No. 61/145,867, filed on Jan. 20, 2009.

(51) Int. Cl. A61K 31/519; A61P 25/16

(52) U.S. Cl. 514/252.16

(57) ABSTRACT

The present invention provides a method for alleviating motor complications and motor indications in a patient suffering from moderate to severe Parkinson's disease by administering a composition which provides an effective amount of the compound of Formula I.

### CROSS-REFERENCE TO RELATED APPLICATIONS

This application is based on and claims the priority of U.S. Provisional Patent Application Ser. No. 61/145,867 filed Jan. 20, 2009, which application is incorporated by reference as if fully set forth herein.

### FIELD OF THE INVENTION

This application relates to methods of alleviating one or more motor symptoms and/or motor complications (collectively, the signs and/or symptoms of Parkinson's disease) in a patient suffering from Parkinson's disease, including motor symptoms associated with being afflicted by Parkinson's disease and motor complications arising from therapy administered in connection with treating the disease, for example, dyskinesia, akathisia, decreased “on”-time or increased “off”-time. This application relates also to methods of treating or alleviating one or more signs and/or symptoms of moderate to severe Parkinson's disease in patients receiving concomitant dopaminergic treatment.

### BACKGROUND OF THE INVENTION

Identification of any publication in this section or any section of this application is not an admission that such publication is prior art to the present invention.

Parkinson's disease is characterized by progressive degeneration of the nigrostriatal dopaminergic pathway. The subsequent reduction in striatal dopamine levels is responsible for the loss of fine motor control, or motor impairment, manifested in those suffering from the disease, termed herein motor symptoms associated with Parkinson's disease. Current methodologies for alleviating motor symptoms associated with Parkinson's disease seek to replace dopamine either within the presynaptic terminal, for example, by administration of L-Dopa, directly through stimulation of the postsynaptic D₂ receptors, or by inhibiting metabolism, for example, by administration of monoamine oxidase type B (MAO-B) or catechol-O-methyltransferase (COMT). Long term use of such therapies is often associated with adverse events. For example, long term therapy with L-Dopa (currently the standard of care) is often associated with adverse events, for example, “wearing-off”, “random on-off” oscillations, or dyskinesia. These complications arising from therapy administered to manage Parkinson's disease, termed herein motor complications, often become progressively more severe with continued treatment.

Data has shown that A2a receptors are present in high density in the basal ganglia and are known to be important in the control of fine motor movement. Highly selective A2a antagonists have demonstrated their efficacy in reducing motor symptoms associated with neurodegenerative diseases. Accordingly, compounds which are A2a receptor antagonists may be useful in alleviating motor symptoms associated with Parkinson's disease. U.S. Pat. No. 6,630,475 to Neustadt et al. (the '475 patent, which is incorporated herein by reference in its entirety) describes the preparation of the compound of Formula I,

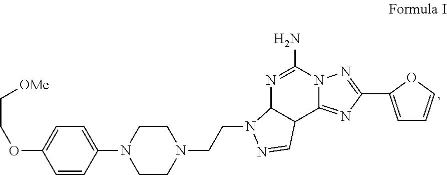

for example, in Schemes 1 to 5, which show general methods of preparing this type of compound and preparative Schemes 1 to 4, which are described beginning at Column 15, line 14, to Column 18, line 25, in conjunction with the procedure described in preparative Scheme 5 beginning at Column 26 line 64 through Column 28, line 39, and in Example 1-83 found in Column 49, which sections are specifically incorporated herein by reference. The '475 patent also describes, in Col. 8, lines 1 to 34, that the compound of Formula I can be prepared as a pharmaceutically acceptable salt, for example, an acid addition salt, which section is also specifically incorporated herein by reference.

### OBJECTIVES AND SUMMARY OF THE INVENTION

What is needed is a method for alleviating motor symptoms associated with Parkinson's disease without incurring the motor complications arising in patients suffering from Parkinson's disease receiving the current standard of care.

This and other objectives and/or advantages are provided by the present invention which in one aspect is a method of alleviating motor symptoms and/or motor complications (collectively motor signs and/or symptoms) associated with Parkinson's disease and/or the treatment thereof, the method comprising administering to a patient in need thereof an amount of a composition which provides to a patient receiving said composition an amount of the compound of Formula I sufficient to provide a plasma concentration in excess of 50 ng/mL.

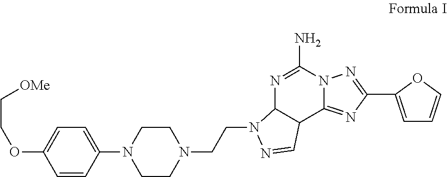

In some embodiments it is preferred to administer an amount of a composition which provides the compound of Formula I in an amount yielding, as a statistical average across a patient population, a Cₘₐₓ of from about 112 ng/mL to about 130 ng/mL. In some embodiments it is preferred to administer an amount of a composition which provides the compound of Formula I in an amount yielding, as a statistical average across a patient population, an AUC_{(tf)} (where AUC_{(tf)} is used herein to indicate exposure at time=infinity, wherein, time at infinity is taken as the time at which the concentration of the compound of Formula I in a serum sample falls below the limit of detection) of from about 336 hr.*ng/mL to about 457 hr*ng/mL. In some embodiments it is preferred to administer an amount of a composition which provides the compound of Formula I in an amount yielding, as a statistical average across a patient population, an AUC(I) measured over a period of T=0 hr. to 1 hr. of from about 352 hr*ng/mL to about 478 hr*ng/mL.

In other aspects, the present invention affords surprising advantages in providing, to a patient receiving concomitant dopaminergic therapy, methods for treating or alleviating one or more signs and/or symptoms of moderate to severe Parkinson's disease by administering to such patients in need thereof an amount of a composition which provides a sufficient amount of the compound of Formula I to improve motor symptoms (for example, motor impairment and loss of fine muscle control) associated with being afflicted by Parkinson's disease and to reduce motor complications associated with receiving dopaminergic therapy. In some embodiments it is preferred to orally administer twice a day (BID) an amount of a composition which provides to a patient in need thereof, 5 mg of the compound of Formula I.

In some embodiments it is preferred to orally administer BID a composition which provides to a patient receiving the composition the compound of Formula I in an amount of at least 2 mg, preferably to administer orally BID an amount of the composition which will provide to the patient receiving the composition the compound of Formula I an amount of from about 2 mg to about 10 mg, more preferably to orally administer BID a composition which provides to a patient receiving the composition the compound of Formula I in an amount of 5 mg.

In some embodiments, an amount of a composition is selected which provides to a patient receiving the composition the compound of Formula I in an amount selected to provide a decrease in mean “Unified Parkinson's Disease Rating Scale (herein, the UPDRS, a scale developed to quantify the signs and symptoms associated with Parkinson's disease, as reported by Olanow, CW; Watts, R. L.; and Koller, W. C. in “An Algorithm (decision tree) For The Management of Parkinson's Disease (2001): Treatement Guidelines”; Neurology, 2001; 56 (11 suppl.): S1-S88, and as reported by Fahn, S.; Elton, R. L.; UPDRS Development Committee, Unified Parkinson's Disease Rating Scale appearing in: Fahn, S; Marsden, C. D.; and Goldstein, M. (eds.), Recent Developments in Parkinson's Disease, Florham Park, N.J.: published by Macmillian; (1987), pp 153 to 163). In some embodiments it is preferred to administer a composition which provides to a patient receiving the composition the compound of Formula I in an amount that, within 2 weeks following commencing administration of the composition, decreases the mean UPDRS Part III—Motor Examination (UPDRS III) score by at least about 3 points below a baseline score established for said patient prior to commencing administration of the composition.

In some embodiments, the alleviation of motor complications associated with Parkinson's disease is manifest by a reduction in average daily “Off-time” experienced by a patient suffering from Parkinson's disease. In some embodiments, the alleviation of motor complications associated with Parkinson's disease is manifest by an increase in average daily “On-time” experienced by a patient suffering from Parkinson's disease. In some embodiments, the alleviation of motor complications associated with Parkinson's disease is manifest by a reduction in average daily “Off-time” and concomitant increase in average daily “On-time” experienced by a patient suffering from Parkinson's disease. In some embodiments it is preferred to administer an amount of a composition providing the compound of Formula I in an amount which yields a decrease in average daily “Off-time” and concomitantly an increase in average daily “On-time” without increasing proportionately “On-time” periods of dyskinesia or troublesome dyskinesia. In some embodiments it is preferred to administer concomitantly with the administration of dopaminergenic treatment a composition providing an amount of the compound of Formula I effective to alleviate or treat one or more motor symptoms associated with Parkinson's disease and/or motor complications associated with receiving dopaminergenic treatment. In some embodiments it is preferred to administer an amount of a composition providing the compound of Formula I which is sufficient to alleviate or treat one or more signs and/or symptoms associated with Parkinson's disease.

The present invention is based in part upon the surprising finding that administration of a composition providing an effective amount of the compound of Formula I to patients with moderate to severe Parkinson's disease both significantly improved patient's average daily “On-time” without troublesome dyskinesia and significantly reduced patient's average daily “Off-time”.

### BRIEF DESCRIPTION OF THE FIGURES

FIG. 1. presents a graphic representation of the relative change in “On”-time and “Off”-time in patients receiving L-Dopa treatment who were also treated with various dosage amounts of a composition providing the compound of Formula I.

FIG. 2 presents a graphic representation of the relative changes in total daily sleep time in patients receiving L-Dopa treatment who were also treated with various dosage amounts of a composition providing the compound of Formula I.

FIG. 3 presents a graphic representation of the mean change in UPDRS III scores 2 hours post dose in patients receiving L-Dopa treatment who were also treated with various dosage amounts of a composition providing the compound of Formula I

FIG. 4 presents a graphic representation of a method for mitigating akathisia using a composition providing the compound of Formula I.

FIG. 5 presents a graphic representation of the percentage of A2a receptor sites occupied with increasing serum concentrations of the compound of Formula I as determined by PET scan.

### DETAILED DESCRIPTION OF THE INVENTION

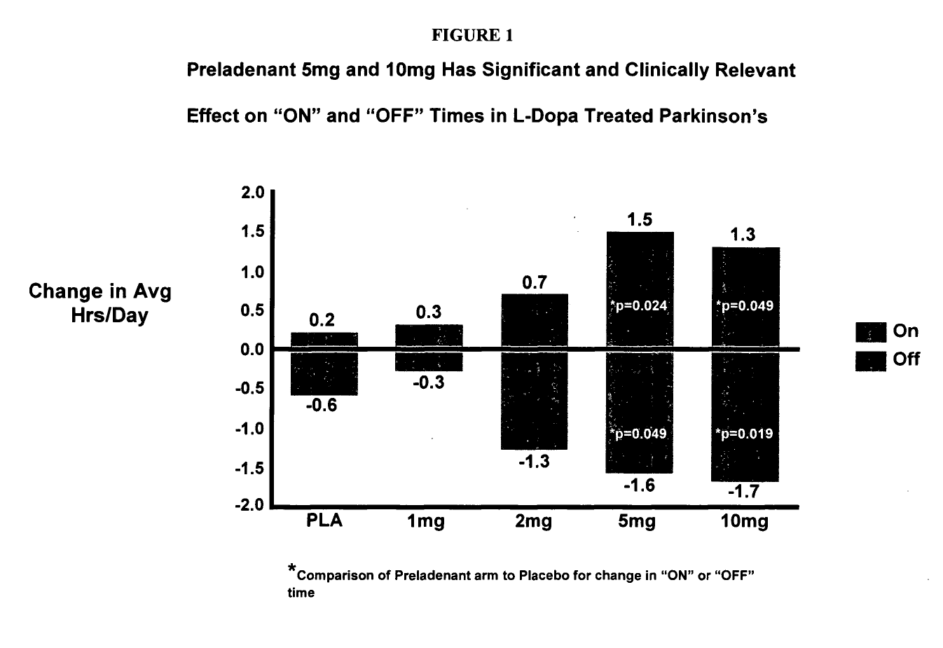

As described by C. D. Marsden in “Parkinson's Disease”, Lancet (1990) 335; pp. 948-952, in most patients with established Parkinson's disease, in addition to the impaired motor function which is a symptom of a patient afflicted with Parkinson's disease, prolonged treatment with L-Dopa (dopaminerginic treatment) also produces characteristic motor complications manifest in a patient's motor functioning which are associated with receiving dopaminerginic treatment for the disease. Examples of motor complications include “Off-time”, where the patient has absent or poor motor function, alternating with “On-time”, which are periods of improved motor function, which may or may not be accompanied by troublesome dyskinesia. Collectively, the motor symptoms associated with being afflicted with Parkinson's disease and the motor complications associated with receiving dopaminerginic therapy for the disease have been referred to herein as “signs and/or symptoms associated with Parkinson's disease”. With reference to FIG. 1, the inventors have surprisingly found that administration of a composition which provides to a patient afflicted with Parkinson's disease an effective amount of the compound of Formula I can be used to alleviate one or more signs and/or symptoms associated with Parkinson's disease arising either from impairment in motor function associated with being afflicted with the disease and/or arising from motor complications associated with receiving prolonged dopaminerginic treatment, for example, L-dopa therapy. As shown in FIG. 1, the administration of a composition providing to a patient the compound of Formula I (described above) in an amount that provides a serum level of at least about 50 ng/mL, preferably a composition administered BID which provides at least about 2 mg of the compound of Formula I, more preferably provides from about 2 mg to about 10 mg of the compound of Formula I, and more preferably which provides about 5 mg of the compound of Formula I can alleviate at least one of the aforementioned motor symptoms associated with being afflicted with Parkinson's disease and/or motor complications associated with receipt of dopaminerginic treatment in patients suffering from Parkinson's disease, that is, alleviate one or more of the signs and/or symptoms of Parkinson's disease.

The data in FIG. 1 was acquired by interviewing patients with moderate to severe Parkinson's disease who had received either a placebo or a dosage of a composition providing the compound of Formula I in addition to L-dopa therapy. These data demonstrate that administration of a composition providing an effective amount of the compound of Formula I to patients afflicted with Parkinson's disease provides the patients with a decrease in average daily “Off-time” periods and an increase in average daily “On-time” periods (as the terms “On-time” and “Off-time” are defined above). From studies of healthy human volunteers, it is believed that oral administration of a composition which provides about 5 mg of the compound of Formula I yields PK parameters based on observed serum levels of the compound of Formula I, expressed as an average value across a population of healthy human volunteers receiving the composition, corresponding to a Cₘₐₓ of from about 112 ng/mL to about 130 ng/mL, and an AUC_{(tf)} of from about 336 hr*ng/mL to about 457 hr*ng/mL. The AUC_{(tf)} PK parameter refers to AUC calculated at t=infinity, which is taken as the time at which the serum level of the compound of Formula I drops below the detectable limit. It is also believed that oral administration to healthy human volunteers of a composition providing about 5 mg of the compound of Formula I is associated with AUC values measured from t=0 to 1 hr. of from about 352 hr*ng/mL to about 478 ng*hr/mL.

As will be seen from the example below and from the information in FIGS. 1 to 3, beneficial effects are surprisingly achieved in patient populations afflicted with motor symptoms associated with Parkinson's disease and experiencing motor complications while on L-dopa therapy when a composition providing the compound of Formula I is administered orally in two divided doses in an amount which provides at least about 5 mg/dose, that is when the composition is administered BID as a dose comprising a composition supplying 5 mg of the compound of Formula I.

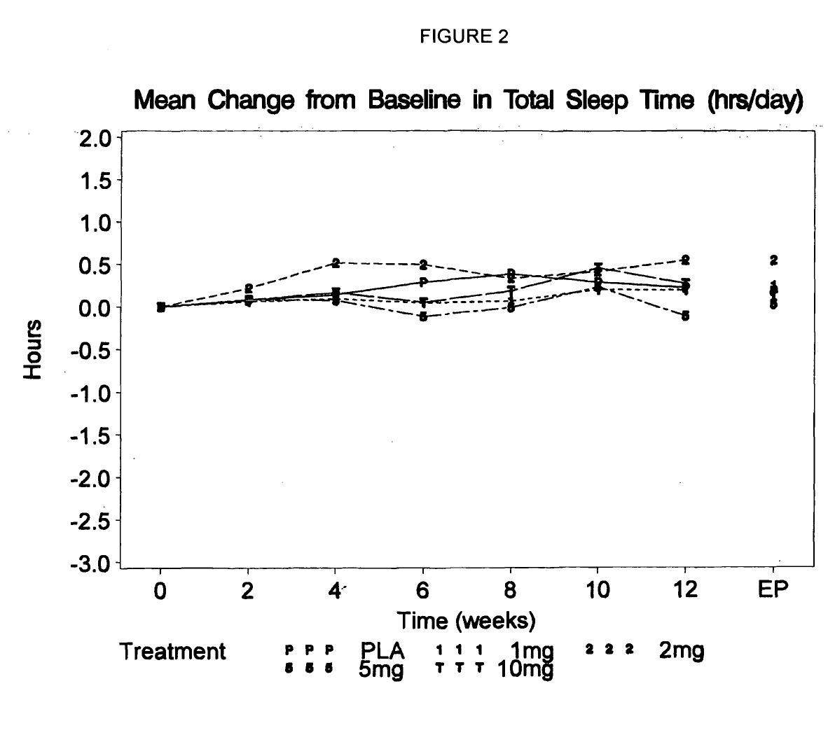

In addition, with reference to FIG. 2, administration of a composition providing the compound of Formula I in an amount of from about 2 mg BID to about 10 mg BID does not significantly adversely effect patient sleep patterns. This is to say that providing an effective amount of the compound of Formula I to patients in need thereof is not associated with increased sleep time. Moreover, patient assessment by clinicians indicates that administration of an a composition providing an effective amount of the compound of Formula I is not associated with induction of sleep attacks in patients to whom it is administered.

in, a composition which provides the compound of Formula I refers to a composition comprising one or more pharmaceutical excipients together with a chemical compound or compounds which when ingested by a patient will provide a beneficial form of the compound of Formula I to the serum of said patient. Accordingly, this may be the free base compound of Formula I, or a pharmaceutically acceptable salt of the compound of Formula I, for example, as described in the '475 patent in Col. 8, lines 1 to 34, and in Col. 95, line 57 to Col. 97, line 52, which sections are specifically incorporated herein by reference.

It will be appreciated that the compositions described herein which provide the compound of Formula I to a patient to whom they are administered can be coadministered with dopaminergic therapy, for example, coadministered with L-dopa alone or in combination with one or more other therapeutic compounds, for example, an MAO-B inhibitor or a COMT inhibitor. Coadministration, as used herein, is contemporaneous, simultaneous, or sequential administration of more than one composition, as well as administration of a single composition comprising more than one therapeutically active substance.

### EXAMPLE

Pharmaceutical Formulation Providing the Compound of Formula I

A composition which would provide the compound of Formula I was prepared for use in clinical trials in accordance with the following process. A dry-blended composition containing the free-base compound of Formula I was prepared by prescreening lactose monohydrate, anhydrous citric acid, croscarmellose sodium and a free base form of the compound of Formula I made in accordance with the sections of the '475 patent referenced above. The screened materials were dry-blended and the blended composition was combined with aliquots of prescreened magnesium stearate and an additional amount of prescreened lactose monohydrate. This mixture was dry-blended and the blended composition was placed into capsules in an amount which contained the desired quantity of the compound of Formula I, which for use in the studies described below was an amount of the composition containing either 1 mg or 5 mg of the free-base compound of Formula I. The weights of the constituents contained in each 1 mg or 5 mg capsule prepared in accordance with this process are reported in Table I, below.

TABLE 1
Compositions providing 1 mg and 5 mg of the compound of Formula I
contained in Capsules

| | | Concentration per Capsule (mg) | |
|---|---|---|---|
| Component | Function | 1 mg Capsule | 5 mg Capsule |

Capsule Fill

| | | | |
|---|---|---|---|
| Compound of | Drug | 1.0 | 5.0 |
| Formula I Micronized | substance | | |
| Lactose | Filler | 375.6 | 366 |
| Monohydrate | | | |
| Croscarmellose | Disintegrant | 20 | 20 |
| Sodium | | | |
| Anhydrous Citric Acid | Acidifier | 1.4 | 7 |
| Magnesium Stearate | Lubricant | 2 | 2 |

| | | |
|---|---|---|
| Theoretical Capsule Fill Weight | 400 | 400 |

Capsule Shell

| | | | |
|---|---|---|---|
| Hard Gelatin | Contain | — | — |
| Capsule^{a} | capsule fill | | |

| | | |
|---|---|---|
| Theoretical Total Capsule Fill Weight | 400 | 400 |

^{a}: Number 1, blue opaque, preservative-free, two-piece hard gelatin capsules.

### Process for Providing Capsules Containing 1 mg of the Compound of Formula I

The manufacturing processes for preparing a composition for filling capsules containing the equivalent of 1 mg of the compound of Formula I (1 mg strength Capsules) is depicted in the flow diagram presented in Diagram 1. In general, the blending process produces multi-kilogram quantities of the composition which is filled into capsules in 400 mg fill weight.

With reference to Diagram 1, 1-mg strength Capsules were manufactured as follows:
Step 1 Lactose monohydrate, anhydrous citric acid, and croscarmellose sodium were passed through an appropriate sized screen and a portion of the screened lactose monohydrate was retained that was approximately five times the quantity (weight) of the magnesium stearate to be added in Step 10.
Step 2 A portion of the screened material from Step 1 was retained for hand sieving with the compound of Formula I in Step 5.
Step 3 A second portion of the screened material from Step 1 was retained for rinsing the container holding the compound of Formula I prior to hand screening (Step 5).
Step 4 A third portion of the screened material from Step 1 was retained for rinsing the screen used to screen the compound of Formula (API) after addition of the screened API to the blender in Step 7.
Step 5 The portion of the screened material retained in Step 2 and the API was transferred to a stainless steel container, and the container which held the API was rinsed with the screened material from Step 3 and added to the stainless steel container.
Step 6 The mixture in the stainless steel container from Step 5 was passed through an appropriately sized screen.
Step 7 The screen from Step 6 was rinsed afterward using the material from Step 4.
Step 8 The remainder of the material from Step 1, the screened material from Step 6, and the rinse material from Step 7 was charged into an appropriately sized diffusion-type blender.
Step 9 The mixture prepared in Step 8 was blended in the blender by operating the blender for approximately 400 revolutions.
Step 10 The lactose monohydrate retained in Step 1 and the magnesium stearate was preblended.
Step 11. The pre-blend composition from Step 10 was passed through an appropriately sized screen.
Step 12. The screened material from Step 11 was added into the blender containing the material from Step 9 and the mixture was blended for approximately 60 revolutions.
Step 13 The blended composition from Step 12 was filled into No. 1 blue opaque, preservative-free, two-piece hard gelatin capsules using a suitable capsule filling machine and the filling process was monitored by assaying the capsule weight. Appropriate adjustments were made to the filling equipment as needed throughout the encapsulation process to achieve the target in-process capsule weight specification range.
Step 14 Capsules filled in Step 13 were polished using suitable polishing equipment and the polished capsules were packaged in a suitable container/closure for later use in clinical trials.

### Process for Providing Capsules Containing 5 mg of the Compound of Formula I

The manufacturing processes for preparing a composition for filling capsules containing the equivalent of 5-mg of the compound of Formula I (5-mg strength Capsules) is depicted in the flow diagram presented in Diagram 2. In general, the blending process produces multi-kilogram quantities of the composition which is filled into capsules in 400 mg fill weight.

With reference to Diagram 2, 5-mg strength Capsules were manufactured as follows:
Step 1 A batch quantity of the compound of Formula I, free base (API), anhydrous citric acid, croscarmellose sodium, and a portion of the batch quantity of lactose monohydrate were passed through an appropriately sized screen and placed into a diffusion mixer tumble blender.
Step 2. The screened ingredients from Step 1 were blended by operating the diffusion mixer tumble blender for approximately 400 revolutions.
Step 3. The remainer of the lactose monohydrate not combined with other excipients in Step 2 was pre-blended with the batch quantity of magnesium stearate and the blended composition was passed through an appropriately sized screen.
Step 4. The screened material from Step 3 was added into the blender containing the material from Step 2 and the mixture was blended by operating the blender for approximately 60 revolutions.
Step 5. The composition from Step 4 was filled into No. 1 blue opaque, preservative-free, two-piece hard gelatin capsules using a suitable capsule filling machine. During the filling operation the capsule fill weight was monitored and the necessary process adjustments were applied throughout the encapsulation process to achieve the in-process capsule weight specification range.
Step 6. The filled capsules from Step 5 were polished using suitable capsule polishing equipment and the polished capsules were placed in a suitable container/closure for later use in clinical trials.

### Cinical Trial in Human Patients

A clinical trial was carried out using 253 patients suffering from Parkinson's disease and receiving L-dopa therapy. Patients had a median Mini-Mental State Examination score of 29 points and were affected with moderate to severe Parkinson's disease, ranking 2.5 (43%), 3 (45%), or 4 (12%) on the Hoehn and Yahr Staging scale.

These patients were continued on their L-dopa therapy and randomized to receive in addition either a placebo (n=49), a composition administered orally BID in an amount providing 1 mg of the compound of Formula I (n=49), a composition administered orally BID in an amount providing 2 mg of the compound of Formula I (n=49), a composition administered orally BID in an amount providing 5 mg of the compound of Formula I (n=49), or a composition administered orally BID in an amount providing 10 mg of the compound of Formula I (n=57). This treatment was continued for 12 weeks while the patients kept a diary recording the “Off-time” periods, “On-time” periods (recording whether “On-time” was accompanied by dyskinesia or troubling dyskinesia) and their total amount of sleep time. These patients were also interviewed clinically and were evaluated as well in accordance with the UPDRS rating scale, as well as for when, and the duration of, sleep attacks experienced during the study. Blood chemistry was followed clinically at regular intervals. The investigators found that reports of common adverse events: Parkinsonism; somnolence; and dyskinesia in the study group occurred with no more frequency than was reported in the placebo group. the investigators surprisingly found improvement in periods of “On-time” and a decrease in periods of “Off-time” with no rise in the proportion of “On-time” with dyskinesia or other motor complications among the group receiving therapeutically effective amounts of the composition providing the compound of Formula I, for example, in a dosage amount providing at least about 2 mg of the compound of Formula I administered BID. Accordingly, this study shows that administration of a composition providing an effective amount of the compound of Formula I can alleviate the signs and symptoms of Parkinson's disease in patients afflicted with Parkinson's disease.

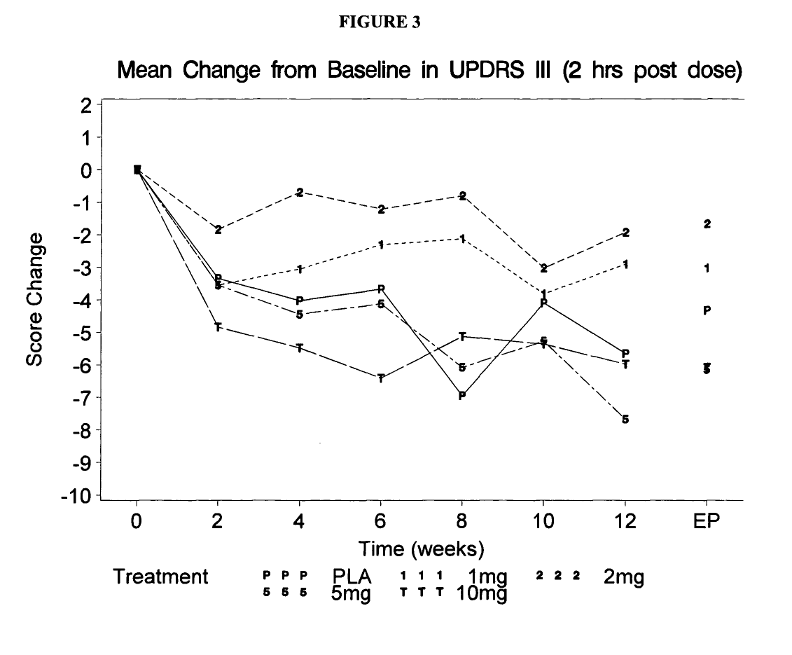

Moreover, with reference to FIG. 3, assessment of patients was made using the Unified Parkinson's Disease Rating Scale (UPDRS) following several weeks of administration. The results of this evaluation indicates that when patients are administered amounts of a composition providing the compound of Formula I in an amount sufficient to maintain a steady state serum level of at least about 50 ng/ml, patient UPDRS scores are improved. With reference to FIG. 3, for example, UPDRS, part III scores were improved by showing a decrease of at least about 3 points below the baseline score at the start of the study. Moreover, and surprisingly, this improvement was not accompanied by an increase in UPDRS part IV scores (part IV is related to signs and symptoms of adverse effects of therapeutic treatment). These data indicate that the administration of a composition providing the compound of Formula I can alleviate motor complications in patients suffering from Parkinson's disease.

The results of this study, which are presented in FIGS. 1 to 3 and described above with regard to clinical observation and interview of patients, indicate that when a composition is administered orally BID in an amount which supplies at least about 5 mg of the compound of Formula I to a patient, there is surprisingly an improvement in UPDRS score which is maintained throughout the course of the study. Moreover, surprisingly there is an increase in average daily patient “On-time” periods and a decrease in average daily patient “Off-time” periods which are not accompanied by objectional side effects, for example, dyskinesia, somnolence, or insomnia.

In a separate study with healthy human volunteers, differences in the bioavailability of the compound of Formula I as provided by a composition used in the study of patients afflicted with Parkinson's disease was conducted and it was found that no statistically significant differences in bioavailability were observed between a study group divided by age or sex as determined by measurement of AUC or Cₘₐₓ in the test subjects.

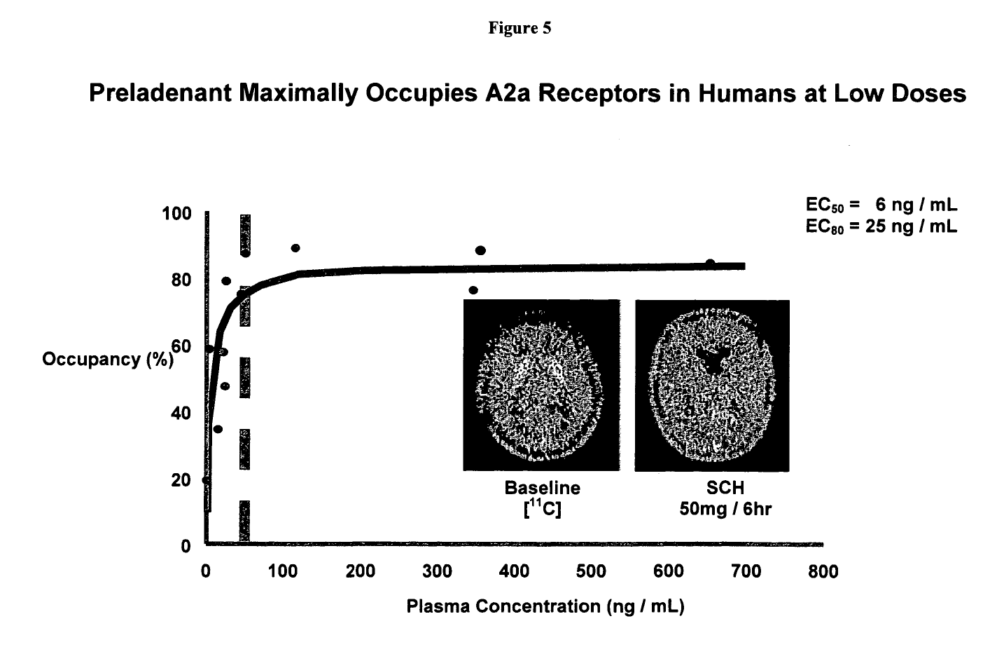

In a separate study with healthy human volunteers, and with reference to FIG. 5, PET scans of a human brain were obtained after IV administration to a subject of a composition intravenous administration of the compound of Formula II which had been labeled with ¹¹C-radio-isotope and then after an interval subsequent intravenous administered the compound of Formula I.

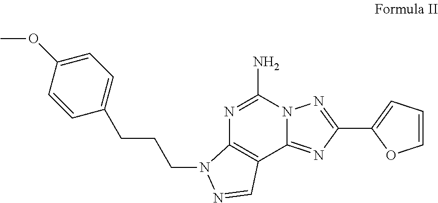

The PET scans indicate high concentrations of the compound of Formula I in regions of the brain known to contain high levels of A2a receptors. This study was carried out using 18 subjects. Capsules containing a composition providing the compound of Formula I (unlabelled) in amounts of 10, 50, or 200 mg were also administered to a subset of test subjects prior to injection of the radio-labeled compound of Formula II. Using the information obtained from the PET scan regarding the extent, location, and inhibition of radiotracer binding permitted calculation of binding potentials for the compound of Formula I. The test subjects were also evaluated for plasma levels of the compound of Formula I during the study. Accordingly, it is believed that a high level of receptor site occupancy is available at plasma concentration levels exceeding about 25 ng/mL in humans. These data indicate that 50% receptor site occupancy is available on average at plasma concentration of 6 ng/ml, while nearly 90% occupancy is available at plasma concentrations in excess of about 50 ng/ml, believed to be Eₘₐₓ for the compound of Formula I (maximum receptor site occupancy). These studies also indicate that the compound of Formula I has a selectivity for the A₂ₐ receptor site of 20,000:1 relative to binding at A₁, A_{2b}, and A₃ receptor sites. This study is consistent with a therapeutic level of plasma concentration being provided by administering a composition orally which provides a human subject to whom it is administered with from at least about 2 mg of the compound of Formula I to about 10 mg of the compound of Formula I and preferably 5 mg of the compound of Formula I. It is believed that BID dosing of a composition providing from about 5 mg to about 50 mg of the compound of Formula I will provide at least about 50% receptor site occupancy for at least about 12 hours/day in at least about 80% of the human population and at least about 80% receptor site occupancy for at least about 18 hours/day in at least about 75% of the population.

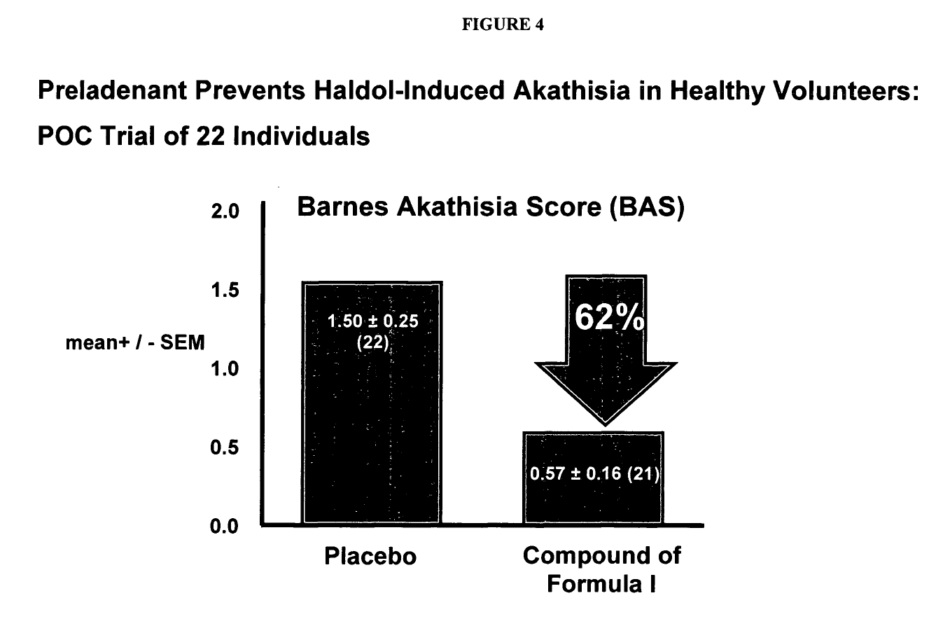

It is believed that a composition which provides an effective amount of the compound of Formula I will also prevent feelings of restlessness, of example, Haldol-induced akathesia. With reference to FIG. 4, administration of an amount of a composition providing about 10 mg of the compound of Formula I to healthy volunteers was successful in suppressing akathisia induced by administration of Haldol.

The above description of the invention is intended to be illustrative and not limiting. Various changes or modifications in the embodiments described herein may occur to those skilled in the art. These changes can be made without departing from the scope or spirit of the invention

1. A method of alleviating one or more signs and/or symptoms in a patient afflicted with Parkinson's disease, the method comprising administering to said patient a composition in an amount providing an amount of the compound of Formula I sufficient to provide a plasma concentration in excess of 50 ng/mL, 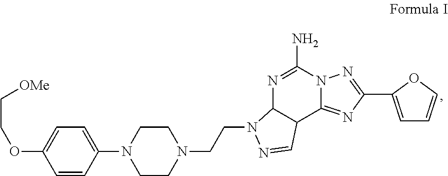
wherein optionally the composition is administered in conjunction with one or more dopaminergic therapeutic agents, and optionally the composition administration is repeated periodically over a period exceeding 3 weeks.

2. The method of claim 1 wherein the amount of the composition administered provides an amount of the compound of Formula I sufficient to yield a Cₘₐₓ of from about 112 ng/mL to about 130 ng/mL and optionally the entire amount of composition to be administered in one 24 hour period is divided into two equal amounts and administered twice daily (BID).

3. The method of claim 1 wherein the amount of the composition administered provides an amount of the compound of Formula I sufficient to yield an AUC_{(tf)} of from about 336 hr*ng/mL to about 457 hr*ng/mL.

4. The method of claim 1 wherein the amount of the composition administered provides an amount of the compound of Formula I which is greater than about 2 mg and optionally the entire amount of composition to be administered in one 24 hour period is divided into two equal amounts and administered twice daily (BID).

5. The method of claim 4 wherein the amount of the composition administered provides an amount of the compound of Formula I of from about 2 mg to about 10 mg and optionally the entire amount of composition to be administered in one 24 hour period is divided into two equal amounts and administered twice daily (BID).

6. The method of claim 1 wherein the amount of the composition administered provides an amount of the compound of Formula I of about 5 mg and optionally the entire amount of composition to be administered in one 24 hour period is divided into two equal amounts and administered twice daily (BID).

7. (canceled)

8. The method of claim 1 wherein alleviating at least one said sign and/or symptom is manifested by: (i) a decrease in patient average daily “Off-time”: or (ii) an increase in patient average daily “On-time”.

9. The method of claim 2, wherein alleviating at least one said sign and/or symptom is manifested by: (i) a decrease in patient average daily “Off-time”; or (ii) an increase in patient average daily “On-time”

10. The method of claim 1 wherein alleviating at least one said sign and/or symptom is manifested by an increase in patient average daily “On-time” with a concomitant decrease in patient average daily “Off-time”.

11. The method of claim 10 wherein alleviating at least one said sign and/or symptom is not accompanied by an increase in periods of dyskinesia.

12. The method of claim 1 wherein the composition administered provides an amount of the compound of Formula I sufficient to provide a decrease in mean UPDRS III score of at least about 3 points below a baseline established for a patient prior to administration of the composition within 2 weeks after beginning administration of the composition.

13.-15. (canceled)

16. The method of claim 2 wherein alleviating at least one said sign and/or symptom is manifested by an increase in patient average daily “On-time” with a concomitant decrease in patient average daily “Off-time”.

17. The method of claim 16 wherein alleviating at least one said sign and/or symptom is not accompanied by an increase in periods of dyskinesia.

18. The method of claim 2 wherein the composition administered provides an amount of the compound of Formula I sufficient to provide a decrease in mean UPDRS III score of at least about 3 points below a baseline established for a patient prior to administration of the composition within 2 weeks after beginning administration of the composition.

19. The method of claim 3, wherein alleviating at least one said sign and/or symptom is manifested by: (i) a decrease in patient average daily “Off-time”; or (ii) an increase in patient average daily “On-time”.

20. The method of claim 3, wherein alleviating at least one said sign and/or symptom is manifested by an increase in patient average daily “On-time” with a concomitant decrease in patient average daily “Off-time”.

21. The method of claim 20 wherein alleviating at least one said sign and/or symptom is not accompanied by an increase in periods of dyskinesia.

22. The method of claim 3 wherein the composition administered provides an amount of the compound of Formula I sufficient to provide a decrease in mean UPDRS III score of at least about 3 points below a baseline established for a patient prior to administration of the composition within 2 weeks after beginning administration of the composition.
